(19) United States
(12) Patent Application Publication
(10) Pub. No.: US 2014/0128617 A1
(43) Pub. Date: May 8, 2014

(54) METHOD FOR PRODUCING PYRAZOLYLCARBOXANILIDES

(71) Applicants: Wahed Ahmed Moradi, Monheim (DE); Norbert Lui, Odenthal (DE); Michael Dockner, Koeln (DE)

(72) Inventors: Wahed Ahmed Moradi, Monheim (DE); Norbert Lui, Odenthal (DE); Michael Dockner, Koeln (DE)

(21) Appl. No.: 14/125,475

(22) PCT Filed: Jun. 19, 2012
(86) PCT No.: PCT/EP2012/061739

§ 371 Date: Dec. 11, 2013

Related U.S. Application Data
(60) Provisional application No. 61/499,280, filed on Jun. 21, 2011.

(30) Foreign Application Priority Data
Jun. 21, 2011 (EP) 11170685.9

(51) Int. Cl. C07D 231/14

(52) U.S. Cl. CPC C07D 231/14
USPC 548/374.1

(57) ABSTRACT

The present invention relates to a simplified process for preparing pyrazolylcarboxanilides by reacting pyrazolylcarboxylic esters with anilines in the presence of a base and removing at least one reaction product.

The present invention relates to a process for preparing pyrazolylcarboxanilides by reacting pyrazolylcarboxylic esters with anilines in the presence of a base.

It is known that 3-(difluoromethyl)-1-methyl-N-phenyl-1H-pyrazole-4-carboxanilides have fungicidal properties (cf. for example WO 2003/070705).

Numerous methods of synthesizing pyrazolylcarboxanilides are known from the literature (cf. WO 2006/024388; US 2011/0054183; US 2010/0174094). The currently most commonly practised processes react appropriate pyrazolylcarboxylic acid derivatives, for example pyrazolylcarbonyl halides (e.g. pyrazolylcarbonyl chlorides), with aniline derivatives, for example 3′,4′-dichloro-5-fluorobiphenyl-2-amine in the presence or absence of a base. The disadvantage with the processes described in the prior art is that the pyrazolylcarbonyl chloride used as coupling partner has to be prepared from the pyrazolylcarboxylic ester in two steps. Industrial process design is greatly determined by economic and ecological considerations and each additional step is associated with appreciable costs.

A commercially interesting and simpler route to carboxamides from carboxylic esters is to aminolyse such esters (cf. for example Jerry March, Advanced Organic Chemistry, 4^{th.} Edition, pp. 421-424). However, the direct conversion of unactivated esters with anilines continues to be difficult and of limited utility in practice. For example, the aminolysis of carboxylic esters frequently requires high temperatures (J. Am. Chem. Soc. 1949, 2215) and/or high pressures (Angew. Chem. 1986, 98, 569-570). To be able to perform the reaction on a laboratory scale under milder conditions, strong alkaline organometallic catalysts are used, but they do not tolerate highly functionalized substrates and are also industrially impracticable (J. Am. Chem. Soc. 1955, 469-472; Tetrahedron Lett. 1971, 321-322, J. Org. Chem. 1963, 2915-2917; J. Org. Chem. 1992, 6101-6103). In addition to other catalysts such as cyanides (J. Org. Chem. 1987, 52, 2033-2036) and boron tribromides (Tetrahedron Lett. 1974, 3995), trimethylaluminium has come to the fore. This catalyst does indeed enable amides to be synthesized in good yields and under mild reaction conditions, but is very caustic, pyrophoric and reacts explosively with water and therefore is unsuitable for the large industrial scale.

It is an object of the present invention to provide a process for synthesizing pyrazolylcarboxanilides from pyrazolylcarboxylic esters and anilines which is more economical than prior art processes. The process shall be suitable for practice on a large industrial scale and provide pyrazolylcarboxanilides in high yield and high purity.

This object is achieved by a process for preparing funcidally efficacious pyrazolylcarboxanilides of formula (III)

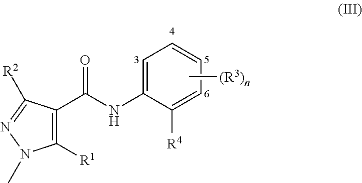

where
R¹ represents hydrogen, fluorine or chlorine,
R² represents methyl, difluoromethyl or trifluoromethyl,
R³ represents hydrogen, fluorine, chlorine, methyl, isopropyl, methylthio or trifluoromethyl,
n represents 1, 2, 3 or 4, preferably 1 or 2 and more preferably 1,
R⁴ represents phenyl optionally substituted one to five times by identical or different substituents selected from halogen, C₁-C₄-alkyl, C₁-C₄-alkoxy, C₁-C₂-haloalkyl or C₂-C₄-haloalkoxy having each 1 to 6 fluorine, chlorine and/or bromine atoms, hydroxyimino-C₁-C₄-alkyl, C₁-C₄-alkoxyimino-C₁-C₄-alkyl, C₁-C₄-haloalkoxyimino-C₁-C₄-alkyl, or in the case of two adjacent substituents, from difluoromethylenedioxy or tetrafluoroethylenedioxy;
   or represents C₃-C10-cycloalkyl or C₃-C10-bicycloalkyl each optionally substituted one to four times by identical or different substituents selected from halogen, C₁-C₄-alkyl, C₁-C₄-haloalkyl and C₃-C10-cycloalkyl,
   or represents unsubstituted (linear or branched) C₁-C₂₀-alkyl, or C₁-C₂₀-alkyl substituted one or more times by identical or different substituents selected from fluorine, chlorine, bromine, iodine and C₃-C₆-cycloalkyl, wherein the cycloalkyl moiety may in turn be optionally substituted one to four times by identical or different substituents selected from fluorine, chlorine, bromine, iodine, C₁-C₄-alkyl and C₁-C₄-haloalkyl,
   or represents C₂-C₂₀-alkenyl or C₂-C₂₀-alkynyl each optionally substituted one or more times by identical or different substituents selected from fluorine, chlorine, bromine, iodine and C₃-C₆-cycloalkyl, wherein the cycloalkyl moiety may in turn be optionally substituted one to four times by identical or different substituents selected from halogen, C₁-C₄-alkyl and C₁-C₄-haloalkyl,
   or combines with the phenyl radical to represent both syn isomers of N-[(1RS,4SR,9RS)-1,2,3,4-tetrahydro-9-(isopropyl)-1,4-methanonaphthalene, or both anti isomers of N-[(1RS,4SR,9SR)-1,2,3,4-tetrahydro-9-isopropyl-1,4-methanonaphthalene,
   or combines with the phenyl radical to represent N-[(1RS,4SR)-1,2,3,4-tetrahydro-9-(dichloromethylene)-1,4-methanonaphthalene,
   or combines with the phenyl radical to represent N-(1,3-dihydro-1,1,3-trimethyl-4-isobenzofuranyl,
      by reacting pyrazolylcarboxylic esters of formula (I)

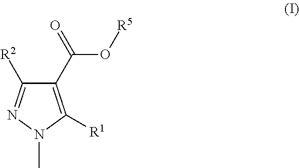

where
R¹ and R² are each as defined above, and
R⁵ represents C₁-C₆-alkyl, C₃-C₈-aryl-C₁-C₆-alkyl, C₁-C₆-alkoxy-C₁-C₆-alkyl, C₁-C₆-thioalkyl, C₁-C₆-alkylthioalkyl, C₁-C₆-alkylsulphonyl-C₁-C₆-alkyl, C₁-C₆-cyanoalkyl, C₁-C₆-haloalkyl, C₁-C₆-nitroalkyl, C₃-C₈-aryl or C₃-C₈-cycloalkyl,
with anilines of formula (II)

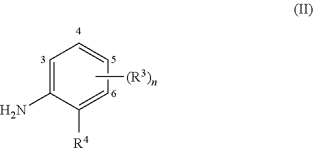

where
R³, R⁴ and n are each as defined above,
wherein the reaction is carried out in the presence of a base and by removing at least one of the reaction products and optionally in an inert solvent.

The pyrazolylcarboxylic esters used as starting materials are generically defined by formula (I). In a preferred embodiment of the present invention, the pyrazolylcarboxylic esters used as starting materials are of formula (I) where
R⁵ represents methyl, ethyl or benzyl.

In a further preferred embodiment of the present invention, the pyrazolylcarboxylic esters used as starting materials are of formula (I) where
R⁵ represents ethyl or methyl.

The anilines used as starting materials are generically defined by formula (II). In a preferred embodiment of the present invention, the anilines used as starting materials are of formula (II) where R³, R⁴ and n are each as defined above and the R³ substituent is in position 5.

In a further preferred embodiment of the present invention, the starting materials used are anilines of formula (II) where
R³ represents hydrogen, fluorine or chlorine,
n represents 1 or 2,
R⁴ represents phenyl optionally substituted two or three times by the same substituent selected from fluorine and chlorine, or represents C₂-C₄-haloalkoxy each having 3 to 6 fluorine atoms and 0 or 1 chlorine atom;
   or represents C₃-bicycloalkyl each optionally substituted one to four times by identical or different substituents selected from halogen and C₁-C₄-alkyl;
   or represents unsubstituted (linear or branched) C₂-C₆-alkyl;
   or combines with the phenyl radical to represent both syn isomers of N-[(1RS,4SR,9RS)-1,2,3,4-tetrahydro-9-(isopropyl)-1,4-methanonaphthalene, or both anti isomers of N-[(1RS,4SR,9SR)-1,2,3,4-tetrahydro-9-isopropyl-1,4-methanonaphthalene;
   or combines with the phenyl radical to represent N-[(1RS,4SR)-1,2,3,4-tetrahydro-9-(dichloromethylene)-1,4-methanonaphthalene;
   or combines with the phenyl radical to represent N-(1,3-dihydro-1,1,3-trimethyl-4-isobenzofuranyl.

In a particularly preferred embodiment of the present invention, the anilines used as starting materials are of formula (II) where
R³ represents 5-fluorine,
n represents 1, and
R⁴ represents 3,4-dichlorophenyl; or
R³ represents hydrogen,
n represents 1, and
R⁴ represents 3,4,5-trifluorophenyl; or
R³ represents hydrogen,
n represents 1, and
R⁴ represents 2-(1,1,2,2-tetrafluoroethoxy), 2-(1,1,2,3,3,3-hexafluoropropoxy) or 2-(3-Cl-1,1,2-trifluoroethoxy); or
R³ represents hydrogen,
n represents 1, and
R⁴ represents 2-(1,1′-bicyclopropyl); or
R³ represents hydrogen,
n represents 1, and
R⁴ represents 2-(1,3-dimethylbutyl); or
R³ represents hydrogen,
n represents 1, and
R⁴ combines with the phenyl radical to represent both syn isomers of N-[(1RS,4SR,9RS)-1,2,3,4-tetrahydro-9-(isopropyl)-1,4-methanonaphthalene or both anti isomers of N-[(1RS,4SR,9SR)-1,2,3,4-tetrahydro-9-isopropyl-1,4-methanonaphthalene; or
R³ represents hydrogen,
n represents 1, and
R⁴ combines with the phenyl radical to represent N-[9-(dichloromethylene)-1,2,3,4-tetrahydro-1,4-methanonaphthalen-5-yl], N-[(1S,4R)-9-(dichloromethylene)-1,2,3,4-tetrahydro-1,4-methanonaphthalen-5-yl] or N-[(1R,4S)-9-(dichloromethylene)-1,2,3,4-tetrahydro-1,4-methanonaphthalen-5-yl]; or
R³ represents hydrogen,
n represents 1, and
R⁴ combines with the phenyl radical to represent N-(1,3-dihydro-1,1,3-trimethyl-4-isobenzofuranyl.

The anilines of formula (II) which are to be used as starting materials to carry out the process of the present invention are also obtainable in situ from the corresponding anilides, for example N-acetanilides.

In a preferred embodiment of the present invention, the fungicidally efficacious pyrazolylcarboxanilides of formula (III) are selected from the group consisting of bixafen, fluxapyroxad, sedaxane, isopyrazam, N-[9-(dichloromethylene)-1,2,3,4-tetrahydro-1,4-methanonaphthalen-5-yl]-3-(difluoromethyl)-1-methyl-1H-pyrazole-4-carboxamide, N-[(1S,4R)-9-(dichloromethylene)-1,2,3,4-tetrahydro-1,4-methanonaphthalen-5-yl]-3-(difluoromethyl)-1-methyl-1H-pyrazole-4-carboxamide, N-[(1R,4 S)-9-(dichloromethylene)-1,2,3,4-tetrahydro-1,4-methanonaphthalen-5-yl]-3-(difluoromethyl)-1-methyl-1H-pyrazole-4-carboxamide, furametpyr, penflufen, 3-(difluoromethyl)-1-methyl-N-[4-(1,1,2,2-tetrafluoroethoxy)phenyl]-1H-pyrazole-4-carboxamide, 3-(difluoromethyl)-N-]4-fluoro-2-(1,1,2,3,3,3-hexafluoropropoxy)phenyl]-1-methyl-1H-pyrazole-4-carboxamide, 3-(difluoromethyl)-1-methyl-N-[2-(1,1,2,3,3,3-hexafluoropropoxy)phenyl]-1-methyl-1H-pyrazole-4-carboxamide and 3-(difluoromethyl)-1-methyl-N-[2-(3-Cl-1,1,2-trifluoroethoxy)phenyl]-1H-pyrazole-4-carboxamide.

Bixafen with the chemical designation N-(3′,4′-dichloro-5-fluoro-1,1′-biphenyl-2-yl)-3-(difluoromethyl)-1-methyl-1H-pyrazole-4-carboxamide and its method of making from known and commercially available components are described in printed publication WO 2003/070705 A.

Fluxapyraxad with the chemical designation 3-(difluoromethyl)-1-methyl-N-(3′,4′,5′-trifluorobiphenyl-2-yl)-1H-pyrazole-4-carboxamide and its method of making from known and commercially available components is described in printed publication WO 2006/087343 A.

Sedaxane, which is a mixture of two cis isomers 2′-[(1RS,2RS)-1,1′-bicycloprop-2-yl]-3-(difluoro-methyl)-1-methylpyrazole-4-carboxanilide and two trans isomers 2′-[(1RS,2SR)-1,1′-bicycloprop-2-yl]-3-(difluoromethyl)-1-methylpyrazole-4-carboxanilide, and its method of making from known and commercially available components are described in printed publications WO 2003/074491 A, WO 2006/015865 A and WO 2006/015866 A.

Isopyrazam, which is a mixture of 2 syn isomers 3-(difluoromethyl)-1-methyl-N-[(1RS,4SR,9RS)-1,2,3,4-tetrahydro-9-isopropyl-1,4-methanonaphthalen-5-yl]pyrazole-4-carboxamide and 2 anti-isomers 3-(difluoromethyl)-1-methyl-N-[(1RS,4SR,9SR)-1,2,3,4-tetrahydro-9-isopropyl-1,4-methano-naphthalen-5-yl]pyrazole-4-carboxamide and its method of making from known and commercially available components are described in printed publication WO 2004/035589 A.

N-[9-(Dichloromethylene)-1,2,3,4-tetrahydro-1,4-methanonaphthalen-5-yl]-3-(difluoromethyl)-1-methyl-1H-pyrazole-4-carboxamide, N-[(1S,4R)-9-(dichloromethylene)-1,2,3,4-tetrahydro-1,4-methanonaphthalen-5-yl]-3-(difluoromethyl)-1-methyl-1H-pyrazole-4-carboxamide and N-[(1R,4S)-9-(dichloromethylene)-1,2,3,4-tetrahydro-1,4-methanonaphthalen-5-yl]-3-(difluoromethyl)-1-methyl-1H-pyrazole-4-carboxamide and their methods of making from known and commercially available components are described in printed publication WO 2007/048556 A.

Furametpyr with the chemical designation 5-chloro-N-(1,3-dihydro-1,1,3-trimethyl-4-isobenzofuranyl)-1,3-dimethyl-1H-pyrazole-4-carboxamide is described in printed publication EP 0315502.

Penflufen with the chemical designation N-[2-(1,3-dimethylbutyl)phenyl]-5-fluoro-1,3-dimethyl-1H-pyrazole-4-carboxamide and its method of making from known and commercially available components are described in printed publication WO 2003/010149 A.

3-(Difluoromethyl)-1-methyl-N-[2-(1,1,2,2-tetrafluoroethoxy)phenyl]-1H-pyrazole-4-carboxamide, 3-(difluoromethyl)-N-[4-fluoro-2-(1,1,2,3,3,3-hexafluoropropoxy)phenyl]-1-methyl-1H-pyrazole-4-carboxamide, 3-(difluoromethyl)-1-methyl-N-[2-(1,1,2,3,3,3-hexafluoropropoxy)phenyl]-1-methyl-1H-pyrazole-4-carboxamide and 3-(difluoromethyl)-1-methyl-N-[2-(3-Cl-1,1,2-trifluoroethoxy)phenyl]-1H-pyrazole-4-carboxamide are described in printed publication WO 2007/017450.

In a particularly preferred embodiment of the present invention, the fungicidally efficacious pyrazolylcarboxanilides of formula (III) are selected from the group consisting of bixafen, fluxapyroxad and isopyrazam.

In a very particularly preferred embodiment of the present invention, the fungicidally efficacious pyrazolylcarboxanilide of formula (III) is bixafen.

It was surprisingly found that pyrazolylcarboxylic esters of formula (I) can be reacted with anilines of formula (II) in the presence of a base, for example sodium methoxide, in organic solvents, for example toluene or NMP or mixtures thereof, to form the corresponding fungicidally efficacious pyrazolylcarboxanilides of formula (III) in good yields. By removing from the reaction mixture at least one of the products present therein in equilibrium, the reaction equilibrium is shifted in the direction of the desired pyrazolylcarboxanilides. It is economically preferable to remove from the reaction mixture at least one alcohol formed in the course of the reaction.

It was further surprisingly found that the acidic difluoromethyl group on the pyrazole is not decomposed by the basic reaction conditions.

In a preferred embodiment of the present invention, the at least one reaction product removed is at least one alcohol. In a particularly preferred embodiment of the present invention, the at least one alcohol is removed by distillation.

A further embodiment of the present invention comprises removing the methanol and ethanol formed in the reaction.

The above-recited general or preferable definitions of radicals and elucidations are combinable with each other in any desired manner; that is to say, combinations between the respective general and preferable ranges are also possible. They apply both to the end products and to the precursor and intermediate products as appropriate. Moreover, individual definitions may also not apply.

When, for example, the starting materials used are 3′,4′-dichloro-5-fluorobiphenyl-2-amine and ethyl 3-(difluoromethyl)-1-methyl-1H-pyrazole-4-carboxylate and also a base and the alcohol formed in the reaction is removed from the reaction mixture, the course of the process according to the present invention can be illustrated by the following scheme (I):

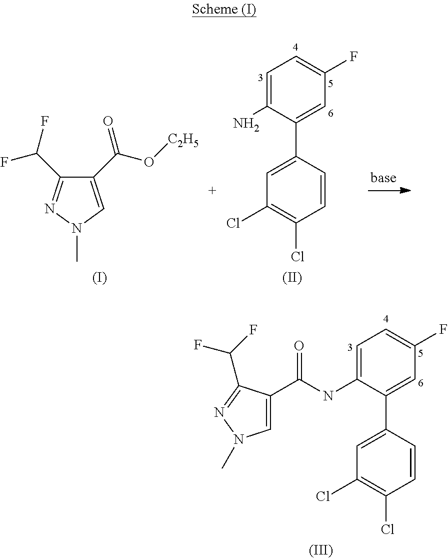

The pyrazolylcarboxylic esters of formula (I) which are needed as starting materials to carry out the process of the present invention are known and/or obtainable by known methods (cf. for example WO 2009/106230; WO 2008/022777).

The anilines of formula (II) which are needed as starting materials to carry out the process of the present invention are likewise known and/or obtainable by known methods such as, for example, hydrogenating the corresponding nitroaromatics (R. C. Larock, Comprehensive Organic Transformations, Wiley-VCH, 2^{nd.} Edition 1999, 821 ff.).

Examples of suitable bases for the present invention are organic bases such as, for example, all amidine/guanidine bases, such as DBU, DBN, pentamethyl- or pentaisopropylguanidine, which may contain no reactive NH groups, and phosphine-imine bases (Schwesinger bases) such as tert-butyliminotris(dimethylamino)phosphorane and 1-tert-butyl-4,4,4-tris(dimethylamino)-2,2-bis[tris(dimethylamino)phosphoranylideneamino]-2λ⁵, 4λ⁵-catenadi(phosphazene). Trialkylamines, which may be alicyclic or open-chain; alkali and alkaline earth metal salts of aliphatic and/or aromatic carboxylic acids, such as acetates, propionates or benzoates; alkali and alkaline earth metal carbonates, bicarbonates, phosphates, hydrogen phosphates and/or hydroxides; and also metal alkoxides, especially alkali or alkaline earth metal alkoxides, for example sodium methoxide, potassium methoxide, sodium ethoxide, magnesium methoxide, calcium ethoxide, sodium tert-butoxide, potassium tert-butoxide or alkali metal isoamoxides. The base is preferably an alkali metal alkoxide selected from the group consisting of sodium methoxide, potassium methoxide, sodium ethoxide, sodium tert-butoxide, potassium tert-butoxide and alkali metal isoamoxide. Sodium methoxide and sodium ethoxide are particularly preferred. The use of sodium methoxide and sodium ethoxide is particularly preferable for economic reasons.

The amount of base needed in the reaction step relative to aniline is simple to determine by a person skilled in the art in routine experimentation. The molar ratio of aniline to base used ranges from 0.01 to 10, more preferably from 0.9 to 2 and even more preferably from 1 to 1.1. The use of larger amounts of base is possible in principle, but disadvantageous for economic reasons.

The process of the present invention is optionally carried out in an inert solvent. Any organic solvent inert under the reaction conditions is possible for performing the process of the present invention.

Examples are ethers, such as ethyl propyl ether, methyl tert-butyl ether, n-butyl ether, anisole, phenetol, cyclohexyl methyl ether, dimethyl ether, diethyl ether, dimethyl glycol diphenyl ether, dipropyl ether, diisopropyl ether, di-n-butyl ether, diisobutyl ether, diisoamyl ether, ethylene glycol dimethyl ether, diglyme, triglyme, 1,2-dimethoxyethane, 1,2-diethoxyethane, 2-ethoxyethyl ether, isopropyl ethyl ether, tetrahydrofuran, methyltetrahydrofuran, dioxane, methyl cyclopentyl ether, tert-amyl methyl ether (TAME), dichlorodiethyl ether and polyethers of ethylene oxide and/or of propylene oxide; acetonitrile, butyronitrile, aliphatic, cycloaliphatic or aromatic hydrocarbons such as pentane, hexane, heptane, octane, nonane and technical grade hydrocarbons, which may be substituted by fluorine and chlorine atoms, such as dichloromethane, trichloromethane, carbon tetrachloride, fluorobenzene, chlorobenzene or dichlorobenzene; for example white spirits with components having boiling points ranging for example from 40° C. to 250° C., cymene, petroleum fractions within a boiling interval of 70° C. to 190° C., cyclohexane, methylcyclohexane, petroleum ether, ligroin, octane, benzene, toluene, xylene, mesitylene, ethylbenzene, cumene, chlorobenzene, bromobenzene, benzotrifluoride, nitrobenzene; dibutyl or ethylene carbonate, dialkyl sulphoxides, N,N-dialkylamides of aliphatic carboxylic acids or of alkylated lactams. Preference is given to solvents selected from the group consisting of tetrahydrofuran, methyltetrahydrofuran, dioxane, methyl cyclopentyl ether, tert-amyl methyl ether (TAME), diglyme, toluene, xylene, mesitylene, cumene, N,N-dimethylacetamide, N,N-dimethylformamide, N-methylpyrrolidone and mixtures thereof. Very particular preference is given to mixtures of NMP and toluene, xylene or cumene. A toluene:NMP mixing ratio of 10:1 is preferred and of 5:1 is particularly preferred.

Solvents are advantageously used in such an amount that the reaction mixture stays readily stirrable throughout the entire process.

In a further embodiment of the present invention, the process of the present invention is carried out without solvent.

The reaction is carried out at a temperature of 20 to 200° C., preferably from 50 to 100° C. and more preferably of 50 to 80° C. and also at a pressure between 1 mbar and 100 bar, and preferably at a pressure between 100 mbar and 600 mbar.

The examples which follow serve to elucidate the process of the present invention without restricting it thereto:

### Preparation Example 1

1. Synthesis of N-(3′,4′-dichloro-5-fluorobiphenyl-2-yl)-3-(difluoromethyl)-1-methyl-1H-pyrazole-4-carboxamide by Removing Methanol and Ethanol

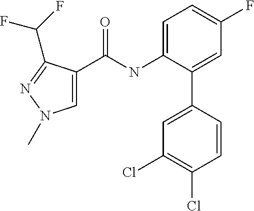 [SMILES: Cn1cc(C(=O)Nc2ccc(F)cc2-c2ccc(Cl)c(Cl)c2)c(C(F)F)n1]

25.20 g [97.39 mmol, 99% GC purity] of 3′,4′-dichloro-5-fluorobiphenyl-2-amine and 20.50 g [99.40 mmol, 99% GC purity] of ethyl 3-(difluoromethyl)-1-methyl-1H-pyrazole-4-carboxylate were dissolved in 70 g of toluene and 15 g of NMP. This solution was admixed, under agitation, with 18.26 g of sodium methoxide (30% by weight in methanol) added in the course of one hour at 70° C. and 500 mbar vacuum. Methanol and ethanol were removed from the reaction mixture as azeotrope with toluene. On completion of the addition of sodium methoxide the reaction mixture was subsequently stirred at 500 mbar and 70° C. for 15 minutes. The vacuum was then reduced to 400 mbar for 15 minutes and to 200 mbar for a further hour. The internal temperature sank to about 60° C. After the reaction had ended, 200 g of water and 100 g of toluene were added at 45° C. to the still stirrable reaction mixture. The product then crystallized out from the tacky mass initially formed. The suspension was adjusted to pH 7 with an HCl solution and cooled down to about 5° C., and the solid material was filtered off, washed with 50 g of water and 50 g of toluene and dried to obtain 36.9 g [83.91 mmol] of N-(3′,4′-dichloro-5-fluorobiphenyl-2-yl)-3-(difluoromethyl)-1-methyl-1H-pyrazole-4-carboxamide as a white solid material having a purity of 94.2% (86.1% yield). ¹H NMR (d₆-DMSO) δ: 9.74 (s, 1H), 8.21 (s, 1H), 7.68 (m, 1H), 7.64 (m, 1H), 7.48-7.46 (m, 1H), 7.40-7.38 (m, 1H), 7.34-7.27 (m, 2H), 7.18 (t, 1H, J=54 Hz), 3.92 (s, 3H).

2. Comparative Example: Synthesis of N-(3′,4′-dichloro-5-fluorobiphenyl-2-yl)-3-(difluoromethyl)-1-methyl-1H-pyrazole-4-carboxamide Without Removing Methanol and Ethanol

6.15 g [24 mmol, 99% GC purity] of 3′,4′-dichloro-5-fluorobiphenyl-2-amine and 5.0 g [24.49 mmol, 99% GC purity] of ethyl 3-(difluoromethyl)-1-methyl-1H-pyrazole-4-carboxylate were dissolved in 17 g of toluene and 3.6 g of NMP. This solution was admixed, under agitation, with 4.5 g of sodium methoxide (30% by weight in methanol) at 80° C. in the course of 15 minutes. On completion of the addition of sodium methoxide the reaction mixture was subsequently stirred at 80° C. for 7 hours. The reaction was tracked via GC analysis to obtain, after about 7 hours, N-(3′,4′-dichloro-5-fluorobiphenyl-2-yl)-3-(difluoromethyl)-1-methyl-1H-pyrazole-4-carboxamide having a GC purity of 66.4%.

### Preparation Example 2

Synthesis of 3-(difluoromethyl)-1-methyl-N-(3′,4′,5′-trifluorobiphenyl-2-yl)-1H-pyrazole-4-carboxamide

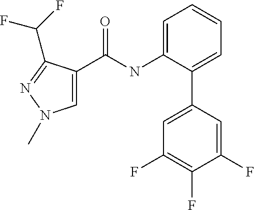 [SMILES: Cn1cc(C(=O)Nc2ccccc2-c2cc(F)c(F)c(F)c2)c(C(F)F)n1]

10 g [44.27 mmol, 98.9% GC purity] of 3′,4′,5′-trifluorobiphenyl-2-amine and 9.04 g [44.27 mmol, 99% GC purity] of ethyl 3-(difluoromethyl)-1-methyl-1H-pyrazole-4-carboxylate were dissolved in 35 g of toluene and 7.5 g of NMP. This solution was admixed, under agitation, with 8.37 g of sodium methoxide (30% by weight in methanol) added at 70° C. and 500 mbar in the course of 20 minutes. Methanol and ethanol were removed from the reaction mixture as azeotrope with toluene. On completion of the addition of sodium methoxide the reaction mixture was subsequently stirred at 500 mbar and 70° C. for 15 minutes. The vacuum was then reduced to 300 mbar for 2 hours and to 200 mbar for a further hour. The reaction was tracked via GC-MS analysis to obtain 3-(difluoromethyl)-1-methyl-N-(3′,4′,5′-trifluorobiphenyl-2-yl)-1H-pyrazole-4-carboxamide having a GC-MS purity of 39%.

### Preparation Example 3

Synthesis of 3-(difluoromethyl)-N-(9-isopropyl-1,2,3,4-tetrahydro-1,4-methanonaphthalen-5-yl)-1-methyl-1H-pyrazole-4-carboxamide

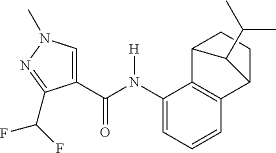 [SMILES: CC(C)C1C2CCC1c1c(NC(=O)c3cn(C)nc3C(F)F)cccc12]

9.86 g [48.98 mmol] of 9-isopropyl-1,2,3,4-tetrahydro-1,4-methanonaphthalen-5-amine (isomer mixture) and 10 g [48.98 mmol] of ethyl 3-(difluoromethyl)-1-methyl-1H-pyrazole-4-carboxylate were dissolved in 35 g of toluene and 7.5 g of NMP. This solution was admixed, under agitation, with 9.08 g of sodium methoxide (30% by weight in methanol) added at 70° C. and 500 mbar in the course of 45 minutes. Methanol and ethanol were removed from the reaction mixture as azeotrope with toluene. On completion of the addition of sodium methoxide the reaction mixture was subsequently stirred at 500 mbar and 70° C. for 15 minutes. The pressure was then reduced to 400 mbar for 15 minutes and to 200 mbar for 1.5 hours. After the reaction had ended, 100 g of water and 50 g of toluene were added to the reaction mixture at 45° C. The organic phase was separated off, the aqueous phase was extracted three times with 50 g of toluene each time, the combined organic phases were dried with Na₂SO₄ and then the solvent was removed in vacuo to obtain 18.2 g of 3-(difluoromethyl)-N-(9-isopropyl-1,2,3,4-tetrahydro-1,4-methanonaphthalen-5-yl)-1-methyl-1H-pyrazole-4-carboxamide as isomer mixture having a GC-MS purity of 71.3%.

1. A process for preparing a funcidally efficacious pyrazolylcarboxanilide of formula (III) 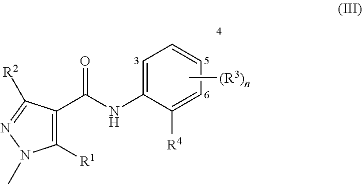
where
R^{l} represents hydrogen, fluorine or chlorine,
R² represents methyl, difluoromethyl or trifluoromethyl,
R³ represents hydrogen, fluorine, chlorine, methyl, isopropyl, methylthio or trifluoromethyl,
n represents 1, 2, 3 or 4, optionally 1 or 2,
R⁴ represents phenyl optionally substituted from one to five times by identical or different substituents selected from the group consisting of halogen, C₁-C₄-alkyl, C₁-C₄-alkoxy, C₁-C₂-haloalkyl or C₂-C₄-haloalkoxy having each from 1 to 6 fluorine, chlorine and/or bromine atoms, hydroxyimino-C₁-C₄-alkyl, C₁-C₄₋alkoxyimino-C₁-C₄-alkyl, C₁-C₄₋haloalkoxyimino-C₁-C₄-alkyl, or in the case of two adjacent substituents, from difluoromethylenedioxy or tetrafluoroethylenedioxy;
or represents C₃-C₁₀-cycloalkyl or C₃-C₁₀-bicycloalkyl each optionally substituted from one to four times by identical or different substituents selected from halogen, C₁-C₄-alkyl, C₁-C₄-haloalkyl and C₃-C₁₀-cycloalkyl,
or represents unsubstituted (linear or branched) C₁-C₂₀-alkyl, or C₁-C₂₀-alkyl substituted one or more times by an identical or different substituent selected from the group consisting of fluorine, chlorine, bromine, iodine and C₃-C₆-cycloalkyl, wherein the cycloalkyl moiety may in turn be optionally substituted from one to four times by an identical or different substituent selected from the group consisting of fluorine, chlorine, bromine, iodine, C₁-C₄-alkyl and C₁-C₄-haloalkyl,
or represents C₂-C₂₀-alkenyl or C₂-C₂₀-alkynyl each optionally substituted one or more times by an identical or different substituent selected from the group consisting of fluorine, chlorine, bromine, iodine and C₃-C₆-cycloalkyl, wherein the cycloalkyl moiety may in turn be optionally substituted from one to four times by an identical or different substituent selected from the group consisting of halogen, C₁-C₄-alkyl and C₁-C₄₋haloalky,
or combines with a phenyl radical to represent both syn isomers of N-[(1RS,4SR,9RS)-1,2,3,4-tetrahydro-9-(isopropyl)-1,4-methanonaphthalene, or both anti isomers of N-[(1RS,4SR,9SR)-1,2,3,4-tetrahydro-9-isopropyl-1,4-methanonaphthalene,
or combines with a phenyl radical to represent N-[(1RS,4SR)-1,2,3,4-tetrahydro-9-(dichloromethylene)-1,4-methanonaphthalene, or combines with a phenyl radical to represent N-(1,3-dihydro-1,1,3-trimethyl-4-isobenzofuranyl,
by reacting a pyrazolylcarboxylic ester of formula (I) 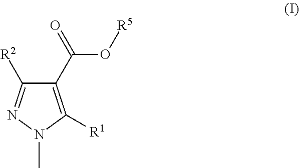
where
R⁵ represents C₁-C₆-alkyl, C₃-C₈-aryl-C₁-C₆-alkyl, C₁-C₆-alkoxy-C₁-C₆-alkyl, C₁-C₆-thioalkyl, C₁-C₆-alkylthioalkyl, C₁-C₆-alkylsulphonyl-C₁-C₆-alkyl, C₁-C₆-cyanoalkyl, C₁-C₆-haloalkyl, C₁-C₆-nitroalkyl, C₃-C₈-aryl or C₃-C₈-cycloalkyl,
with an aniline of formula (II) 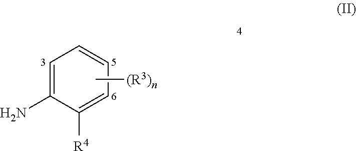
wherein
said reacting is carried out in the presence of a base and by removing at least one reaction product and optionally in an inert solvent.

2. The process according to claim 1, wherein the pyrazolylcarboxylic ester used as a starting material is of formula (I) where
R⁵ represents methyl, ethyl or benzyl.

3. The process according to claim 1, wherein the starting material used comprises an aniline of formula (II) where
R³ represents hydrogen, fluorine or chlorine,
n represents 1 or 2,
R⁴ represents phenyl optionally substituted two or three times by the same substituent selected from fluorine and chlorine, or represents C₂-C₄-haloalkoxy each having from 3 to 6 fluorine atoms and from 0 to 1 chlorine atom;
or represents C₃-bicycloalkyl each optionally substituted from one to four times by an identical or different substituent selected from halogen and C₁-C₄-alkyl;
or represents an unsubstituted (linear or branched) C₂-C₆-alkyl;
or combines with a phenyl radical to represent both syn isomers of N-[(1RS,4SR,9RS)-1,2,3,4-tetrahydro-9-(isopropyl)-1,4-methanonaphthalene, or both anti isomers of N-[(1RS,4SR,9SR)-1,2,3,4-tetrahydro-9-sopropyl-1,4-methanonaphthalene;
or combines with a phenyl radical to represent N-[(1RS,4SR)-1,2,3,4-tetrahydro-9-(dichloromethylene)-1,4-methanonaphthalene;
or combines with a phenyl radical to represent N-(1,3-dihydro-1,1,3-trimethyl-4-isobenzofuranyl.

4. The process according to claim 1, wherein the aniline used as a starting material is of formula (II) where
R³ represents 5-fluorine,
n represents 1, and
R⁴ represents 3,4,5-trifluorophenyl; or
represents 2-(1,1,2,2-tetrafluoroethoxy), 2-(1,1,2,3,3,3-hexafluoropropoxy) or 2-(3-Cl-1,1,2-trifluoroethoxy); or
represents 2-(1,1′-bicyclopropyl); or
represents 2-(1,3-dimethylbutyl); or
combines with a phenyl radical to represent both syn isomers of N-[(1RS,4SR,9RS)-1,2,3,4-tetrahydeo-9-(isopropyl)-1,4-methanonaphthalene or both anti isomers of N-[(1RS,4SR,9sr)-1,2,3,4-tetrahydro-9-isopropyl-1,4-methanonaphthalen; or
combines with a phenyl radical to represent N-[9-dichloromethylene)-1,2,3,4-tetrahydro-1,4-methanonaphthalen-5-yl], N-[(1S,4R)-9-(dichloromethylene)-1,2,3,4-tetrahydro-1,4-methanonaphthalen-5-yl]; or
combines with a phenyl radical to represent N-(1,3-dihydro-1,1,3-trimethyl-4-isobenzofuranyl.

5. The process according to claim 1, wherein the compound of formula (III) is selected from the group consisting of bixafen, fluxapyroxad, sedaxane, isopyrazam, N-[9-(dichloromethylene)-1,2,3,4-tetrahydro-1,4-methanonaphthalen-5-yl]-3-(difluromethyl)-1-methyl-1H-pyrazole-4-carboxamide, N-[(1S,4R)-9-(dichloromethylene)-1,2,3,4-tetrahydeo-1,4-methanonaphthalen-5-yl]-3-(difloromethyl)-1-methyl-1H-pyrazole-4-carboxamide, N-[(1R,4S)-9-(dichloromethylene)-1,2,3,4-tetrahydro-1,4-methanonaphthalen-5-yl]-3-(difluoromethyl)-1-methyl-1H=pyrazole-4-pyrazole-4-carboxamide, furametpyr, penflufen, 3-(difluoromethyl)-1-methyl-N-[2-(1,1,2,2-tetrafluoroethoxy)phenyl]-1H-pyrazole-4-carboxamide, 3-(difluoro-methyl)-N-[4-fluoro-2-(1,1,2,3,3,3-hexafluoropropoxy)phenyl]-1-methyl-1H-pyrazole-4-carbox-amide, 3-(difluoromethyl)-1-methyl-N-[2-(1,1,2,3,3,3-hexafluoropropoxy)phenyl]-1-methyl-1H-pyrazole-4-carboxamide and 3-(difluoromethyl)-1-methyl-N-[2-(3-Cl-1,2-trifluoroethoxy)-phenyl]-1H-pyrazole-4-carboxamide.

6. The process according to claim 1, wherein the compound of formula (III) is selected from the group consisting of bixfen, fluxapyroxad and isopyrazam.

7. The process according to claim 1, wherein the compound of formula (III) is bixafen.

8. The process according to claim 1, wherein the at least one reaction product removed is at least one alcohol.

9. The process according to claim 8, wherein the at least one alcohol is removed by distillation.

10. The process according to claim 1, wherein the base is selected from the group consisting of sodium methoxide, potassium methoxide, sodium ethoxide, sodium tert-butoxide, potassium tert-butoxide and alkali metal isoamoxide.

11. The process according to claim 10, wherein the base is selected from the group consisting of sodium methoxide and sodium ethoxide.

12. The process according to claim 1, wherein the inert solvent is at least one selected from the group consisting of tetrahydrofuran, methyltetrahydrofuran, dioxane, methyl cyclopentyl ether, tert-amyl methyl ether, diglyme, toluene, xylene, mesitylene, cumene, N,N-dimethylacetamide, N,N-dimethylformamide, and N-methylpyrrolidone.

13. The process according to claim 1, wherein said reacting is carried out without solvent.

14. The process according to claim 1, wherein molar ratio of aniline to base is in a range of from 0.01 to 10.

15. The process according to claim 1, wherein the reacting is carried out at a temperature of from 20 to 200° C.
